(19) United States
(12) Patent Application Publication
(10) Pub. No.: US 2005/0130925 A1
(43) Pub. Date: Jun. 16, 2005

(54) Method and composition for enhancing PGE1 production in vascular endothelial and smooth muscle cells

(75) Inventors: Pierre Zoldhelyi, Houston, TX (US); Qi Liu, Houston, TX (US); George Bobustuc, Houston, TX (US); James T. Willerson, Houston, TX (US)

Correspondence Address: CONLEY ROSE, P.C., P. O. BOX 3267, HOUSTON, TX, 77253-3267, US

(73) Assignees: The Board of Regents of the University of Texas System, Austin, TX (US); The Texas Heart Institute, Houston, TX (US)

(21) Appl. No.: 10/972,528

(22) Filed: Oct. 25, 2004

Related U.S. Application Data
(60) Provisional application No. 60/514,080, filed on Oct. 24, 2003.

(51) Int. Cl. A61K 48/00; A61K 31/202

(52) U.S. Cl. 514/44; 514/560

(57) ABSTRACT

Compositions and methods for gene transfer of cyclooxygenase (COX) isoforms alone or in conjunction with administration of one or more fatty acid substrate for the COX isoform (e.g., dihommo-γ-linoleic acid (DGLA)) are disclosed. Methods for enhancing synthesis of the prostaglandins E₁ (PGE₁) and prostacyclin (PGI₂), without marked local production of pro-inflammatory prostaglandin E₂ (PGE₂) are also disclosed. The compositions and methods are valuable for protection of vascular conduits, kidney function, airway patency, and renal, cardiac, and other allografts, and promoting increased vascular flow, mucus secretion and bicarbonate secretion as protective factors against gastric and duodenal ulcers.

### CROSS-REFERENCE TO RELATED APPLICATIONS

This application claims the benefit under 35 U.S.C. § 119(e) of U.S. Provisional Patent Application No. 60/514,080 filed Oct. 24, 2003, the disclosure of which is incorporated herein by reference.

### STATEMENT REGARDING FEDERALLY SPONSORED RESEARCH OR DEVELOPMENT

The work performed during the development of this invention was supported in whole or in part by U.S. Government funds. Accordingly, the U.S. Government has a paid-up license in this invention and the right in limited circumstances to require the patent owner to license others on reasonable terms as provided for by the terms of Grant No. R01 HL073346-01 awarded by the National Institutes of Health.

### BACKGROUND OF THE INVENTION

Cyclooxygenase (COX, also called prostaglandin endoperoxide H synthase) plays a critical role in essential fatty acid metabolism, leading to the biosynthesis of a group of labile bioactive prostaglandins (PGs)¹⁻⁴. Two isoforms of COX enzyme are responsible for this catalytic process⁵⁻⁷; COX-1 and COX-2. COX-1 is constitutively expressed in most mammalian cell types. Whereas COX-2 is generally undetectable under normal physiological condition and is induced by a variety of proinflammatory cytokines and growth factors⁸⁻¹².

The biological importance of COX-1 and COX-2 in maintaining the integrity of gastrointestinal mucosa and kidney blood flow has been extensively studied¹³⁻¹⁶. Moreover, several lines of evidences clearly demonstrate the preventive potential of COX-1 on platelet aggregation, restenosis and cardiovascular malfunctions^{17,18}. But unlike COX-1, COX-2 has been implicated in multiple diseases such as arthritis and tumor angiogenesis¹⁹⁻²².

Prostaglandins generated through COX pathway exert diverse biological activities^{3,23,24}. PGI₂ and PGE₂ serve as potent vasodilators and inhibitors of vascular smooth muscle cell (SMC) proliferation²⁵⁻²⁷. PGI₂ also possesses anti-thrombogenic properties¹⁷. PGE₂ has a housekeeping role in keeping the fluid balance in stomach. However, substantial data have revealed the adverse effects of PGE₂, including its involvement in inflammation and carcinoma formation^{28,29}.

The beneficial aspects of PGs in vascular circulation system (i.e., vasoprotective effects) are attributed in general to the COX-1 dependent production of prostacyclin (PGI₂) by vascular endothelial cells. Therefore, local gene transfer of COX-1 to the injured vascular wall has focused on the increased production of PGI₂, recognizing that enhanced synthesis of PGE₂ could account for potential side effects of COX-1 transfer. Little attention has been paid to the possible enhancement of other potentially beneficial PGs after COX-1 gene transfer in the vascular system and gastrointestinal mucosa.

Prostaglandin E₁ (PGE₁) has multiple favorable vasoprotective actions. It is vasodilative, anti-inflammatory, antithrombotic, antimitotic, antimigratory, and antiangiogenic. Thus, PGE₁, together with PGI₂, have been widely accepted as “good” prostaglandins, as opposed to PGE₂, a potentially “bad” prostaglandin with prothrombotic and strong proinflammatory properties. Previous studies in vascular systems have shown that PGI₂, PGE₂, and PGE₁ share only two important properties, i.e., they are all potent vasodilators and inhibitors of vascular smooth muscle cell proliferation.²⁵⁻²⁷

PGE₁ offers a unique combination of beneficial properties. Unlike PGE₂ and like PGI₂, PGE₁ possesses antithrombotic properties.⁴⁸ In vitro, PGE₁ shows significant antithrombotic activity at concentrations at which PGE₂ shows little or none.^{49,50} PGE₁'s major metabolite, the relatively stable 13,14-dihydro-PGE₁ retains most of the parent compound's antiaggregatory activity.⁵⁰ PGE₁ is a more potent vasodilator than PGE₂.⁵¹ Unlike PGI₂ and PGE₂, PGE, may inhibit collagenase-mediated migration of fibroblasts.⁵² PGE₁ also has potent anti-inflammatory properties when compared with PGE₂.^{53,44} PGE₁ may reduce neointima proliferation after angioplasty and bypass grafts^{45,54} and selectively upregulate, in vitro and in a dose-dependent manner, the expression of human hepatic LDL receptors thereby reducing cholesterol content in the arterial wall.⁵⁵ Clinically, PGE₁ is used as a potent vasodilator in the setting of pulmonary hypertension, and it has recently been tested with good results in clinical trials as a treatment for late-stage peripheral arteriopathy.⁵⁶

Prostaglandins are biosynthesized by COX isoforms from dihommo-γ-linolenic acid (DGLA) and arachidonic acid (AA), both of which are derived from dietary linoleic acid. Linoleic acid is the common precursor of γ-linolenic acid (GLA), which gives rise consecutively to DGLA and AA after specific desaturase (delta-6 and delta-5) and elongase action. Dihommo-γ-linolenic acid is the specific precursor of series 1 prostaglandins (e.g., PGE₁); AA is the specific common precursor of series 2 prostaglandins (e.g., PGI₂, PGE₂) and thromboxane A₂ (TXA₂). Both fatty acids have properties with important implications for vascular systems: DGLA does not give rise to TXA₂ (a potent platelet aggregator and vasoconstrictor), whereas AA, does.⁵⁷

With some notable exceptions (i.e., central nervous tissue, pancreas, kidney, and testis), most human tissues produce much less PGE₁ than PGE₂. This is explained in part by the different constitutive expression and kinetic preferences of COX isoforms: COX-1 prefers AA to DGLA, whereas COX-2 has similar affinities for both. Theoretically, however, COX-1's kinetic preference for AA could be partly overcome if more DLGA were made available, although there are clear limitations as to both the maximal amount of DGLA that could be taken up in the membrane phospholipid layer and the amount of DGLA that could be used by COX-1.^{58,59} Previous studies have shown that, after DGLA or GLA administration, only a small fraction of DGLA is converted to AA^{60,61} because of the limited activity of delta-5 desaturase in humans.

Studies of GLA supplementation in humans^{60,61} have shown that the synthesis of series 1, but not series 2, prostaglandins is selectively elevated. Although the tissue levels of PGE₁ after GLA consumption are still more modest than those of PGE₂, the effects are noteworthy as some of PGE₁'s beneficial biologic effects are ˜20 times stronger than those of PGE₂.^{β}The potential ability of dietary GLA to favorably modulate cardiovascular risk factors has been studied extensively,⁶³ and recent evidence from murine studies indicates that dietary GLA reduces not only the average medial thickness of vessel walls but also the size of atherosclerotic lesions.₆₄

It was previously shown that COX-1 gene transfer in vascular systems induces significant, durable vasodilation that correlates with an early increase in prostacyclin production.³⁰ We hypothesized that the beneficial effects of COX-1 gene transfer included increased PGE₁ production and concomitantly a more favorable PGE₁/PGE₂ ratio. If both PGE₁ and PGE₂ are increased, their overall impact upon the outcome of a vascular response to injury should be determined by the balance between these 2 partially antagonistic prostaglandins (i.e., the PGE₁/PGE₂ ratio). The higher the ratio, the more favorable the profile and the greater the relative suppression of PGE₂ synthesis. To explore this hypothesis, a study was designed to primarily profile PGE₁, PGI₂, and PGE₂ expression after COX-1 gene transfer in vitro and in vivo. In addition, it was investigated whether it would be possible to selectively enhance the local production of PGE₁ (“good” prostaglandin) over PGE₂ (“bad” prostaglandin), without significantly affecting PGI₂ expression, by combining COX-1 gene transfer with administration of dietary amounts of the specific PGE₁ precursor DGLA.

Peptic ulcer disease (PUD) is one of the most common diseases affecting the GI tract. It causes inflammatory injuries in either the gastric or duodenal mucosa, with extension beyond the submucosa into the muscularis mucosa. The normal stomach maintains a balance between the protective factors (i.e., mucus and bicarbonate secretion, blood flow) and aggressive factors (i.e., acid secretion, pepsin). Gastric ulcers develop when aggressive factors overcome the normal protective mechanism.⁷⁰ One of the most important protective factors of the gastric mucosa is PGE₁, which increases the submucosal and mucosal blood flow and stimulates mucus production and bicarbonate secretion, which in turn serve to protect the endothelial gastrointestinal lining against the harmful effects of acid secretion.^{65,66} Misoprostol (Cytotec), a PGE₁ analogue used in clinical practice, was recently demonstrated to have important therapeutic benefits on the gastric mucosa, but its clinical use is seriously limited by its adverse side effects (including diarrhea and abdominal pain observed in 14-40% of patients) and by its cumbersome administration schedule (4 doses/d).^{67,68,69} The ability to have the gastric mucosal cells or submucosal cells produce vasodillatory factors in the presence of a tolerable substrate would be beneficial to protecting the gastric mucosa from PUD.

Recently, intravascular prostaglandin E1 (PGE₁) administration has shown vasoprotective effects superior to PGI₂. Various medicinal formulations of the unstable prostanoid PGE₁ are currently under study as pharmacological vasodilators and inhibitors of restenosis in injured blood vessels. However, administration of PGE₁ requires intravenous or intra-arterial infusion, which limits its administration to relatively short times (minutes to a few hours) and is associated with only short-term effects, risks, discomfort, and side effects similar to systemic administration of prostacyclin. Whether vascular endothelial cells and smooth muscle cells can synthesize PGE₁ is unknown. Nor is it clear whether COX-1 gene transfer enhances the biosynthesis of PGE₁ in animal models.

### SUMMARY OF THE PREFERRED EMBODIMENTS

The effect of COX-1 DGLA and AA dependent metabolic pathways on PGE₁ biosynthesis was comparatively evaluated both in vitro and ex vivo, and it was discovered that COX-1 overexpression significantly increased PGE₁ level (significantly higher when stimulated with DGLA than with AA) in both human aortic endothelial cells (HAEC) and human coronary artery smooth muscle cells (HCASMC). A positive effect of COX-1 on PGI₂ and PGE₂ production was also observed. Additionally, COX-1 was locally delivered to balloon-injured carotid arteries of New Zealand-White Rabbits. Ex vivo data illustrate that COX-1 preferentially stimulated PGI₂ production and meanwhile augmented PGE₁ and PGE₂ level.

This is believed to be the first report of increasing PGE₁ production by cyclooxygenase gene transfer ex vivo and in vitro. Now demonstrated for the first time is the use of gene transfer as a means to enhance PGE₁ synthesis and relatively suppress PGE₂ synthesis in cells and ex vivo. Prior to the present disclosure, it was thought that vascular cells (e.g., smooth muscle cells, endothelial cells) do not produce PGE₁.

While it is known that cyclooxygenase-1 (COX-1) gene transfer in vascular systems enhances prostacyclin (PGI₂) production and promotes durable vasodilation³⁰, it is now demonstrated that COX-1 gene transfer also beneficially increases prostaglandin E₁ (PGE₁) production while relatively suppressing PGE₂ production, resulting in a more favorable PGE₁/PGE₂ ratio, and that this beneficial expression profile is strongly enhanced by dihommo-γ-linolenic acid (DGLA) stimulation. It is disclosed herein that administration and overexpression of the gene encoding cyclooxygenase (COX) isoforms (COX-1 serving as a representative example for other COX isoforms) in combination with dihommo-γ-linoleic acid (DGLA) supplementation of cells and/or administration in vivo enhances the production of PGE₁ in vascular cells and at the same time relatively suppresses synthesis of PGE₂. PGE₂ is known to have, in general, undesirable inflammatory, proatherosclerotic, side effects. The present mode of enhancing production of the desired PGE₁ contrasts with those typically employed in prior investigations in which a gene was administered with a drug. In those studies, the drug was not used to increase the activity of the gene product, but instead attempted to capitalize on the drug causing overexpression of the transfected gene.

In light of the experiments described herein, it is suggested that COX-1 or COX-2 gene transfer has the ability to enhance PGE₁ gene transfer in living cells, with potential benefit for the treatment of vascular stenotic, thrombotic, and inflammatory disease, and, by extension, maintain renal function, and improve or prevent stroke, bronchoconstrictive disease, where PGE₁ will induce bronchodilation via stimulation of adenylyl cyclase and local increase in cyclic AMP, and improve peptic ulcer disease by vasodilation and increasing blood flow, resulting in increased mucus and bicarbonate secretion.

A single stable gene transfer overcomes the need for continuous parenteral infusion of PGE₁ and PGI₂, which is burdensome to the patient, very expensive, and carries side-effects (e.g., flushes, hypotension, infection through the indwelling catheter used for delivery of PGE₁ over weeks to months) as is currently the case for infusion of PGI₂ for pulmonary hypertension, and other diseases.

An advantage of gene transfer compared to drug treatment alone is that of local, instead of general expression a gene product (such as COX-1 or COX-2) or the beneficial product it produces (PGI₂ and PGE₁). Single administration, instead of repeated or continuous intravascular administration of drugs exposes the body to a greater and more generalized load of a) gene vector or means of gene transfer, and b) the desired beneficial gene product, e.g., the COX or PGE₁ synthesizing enzyme (PGES) and its bioactive beneficial products, including the eicosanoids (or more specifically the prostanoids), prostacyclin (PGI₂) and PGE₁. The choice of the gene vector one can use for local or regional administration of the COX gene (as well as other genes) can be tailored to the desired duration of PGE₁ expression and relative PGE₂ suppression.

Accordingly, in certain embodiments of the present invention compositions are provided comprising gene vectors containing cyclooxygenase (COX) isoforms (e.g., COX-1, COX-2) for transfecting a vascular smooth muscle cell (VSMC), endothelial cell (EC), or gastric mucosal or submucosal cell. In some embodiments the compositions also contain a drug that enhances production of PGE₁ in vascular cells or gastric mucosal or submucosal cells. In some embodiments the gene vector also includes cDNAs encoding either related prostaglandins, which further augment PGE₁ or PGI₂, such as PGE synthase (PGES) or prostacyline synthase (PGIS), or phospholipases, which release precursor fatty acids (e.g., DGLA) from the cell membrane. In some embodiments of the present invention, a kit is provided which contains a COX isoform-transducing vector, and one or more substrate fatty acid (e.g., linolenic acid, AA, DGLA) in a pharmaceutically acceptable carrier, suitable for clinical use.

In accordance with further embodiments of the present invention, methods of transducing vascular smooth muscle cells, endothelial cells or gastric mucosal or submucosal cells using the above-described compositions are provided. In certain embodiments the method comprises the gene transfer of cyclooxygenase (COX) isoforms (e.g., COX-1, COX-2) alone. In certain preferred embodiments, the method comprises additionally administering one or more PGE₁ precursor fatty acid that can serve as a substrate for the transduced COX isoform, including but not limited to, DGLA. In some embodiments, the method includes transferring the COX isoform-containing vector with another drug that enhances the production of PGE₁ in transduced vascular cells. In still other embodiments the method includes transfecting vascular cells, gastric mucosal cells, or gastric submucosal cells with cDNAs encoding either related synthase genes, which encode proteins that further augment PGE₁ or PGI₂, such as PGE synthase (PGES) or prostacyline synthase (PGIS), or phospholipases, which release precursor fatty acids (including DGLA) from the cell membrane to enhance PGE₁ and PGI₂ synthesis.

The present disclosure is also believed to be the first report that COX-1 gene transfer advantageously increases PGE₁ production while relatively suppressing PGE₂ production, to provide a more favorable PGE₁/PGE₂ ratio, and that this advantageous expression profile is strongly enhanced by DGLA stimulation.

Accordingly, in certain preferred embodiments of the present invention, a method is provided in which PGE₁ synthesis is selectively enhanced in vascular cells or gastric mucosal or submucosal cells by COX gene transfer, as described above, together with supplemental administration of a fatty acid substrate for the COX enzyme. Supplementation preferably includes administering to the individual an effective amount of one or more fatty acid substrate such as, for example, DGLA. Suitable modes of administration of the fatty acid are known in the art. In some embodiments, a method of enhancing production of PGE₁ in vascular cells (e.g., vascular endothelial or smooth muscle cells) or in gastric mucosal or submucosal cells is provided which includes introducing a recombinant cDNA encoding at least one cyclooxygenase isoform into the vascular cells, such that vascular cells overexpress said cyclooxygenase isoform; and treating the resulting overexpressing cells with an amount of at least one fatty acid substrate for the COX isoform, whereby production of PGE₁ by the cells is enhanced. For example, in certain preferred embodiments a concentration of at least 20 μM dihommo-γ-linolenic acid (DGLA) is established in the cells or in their immediate environment. In some embodiments, the concentration of DGLA is in the range of 50-100 μM. Together, COX gene transfer and PGE₁ precursor fatty acid administration results, ex vivo and in vitro, in a highly favorable prostaglandin expression profile in vascular systems. This profile is characterized by increased PGE₁ and PGI₂ production and concomitant relative suppression of PGE₂.

Also provided in accordance with certain embodiments of the present invention is a method of treating vascular tissue in vivo is provided which includes transducing vascular endothelial cells and/or vascular smooth muscle cells using an above-described composition or method, supplemented by administration of a PGE1 precursor fatty acid, whereby suppression of synthesis of PGE₂, a pro-inflammatory eicosanoid, results.

Still further provided in accordance with certain embodiments of the present invention is a method of treating gastric mucosa in vivo. This method includes transducing cells of the gastric mucosa and/or gastric submucosa cells using an above-described composition or method, supplemented by administration of a PGE₁ precursor fatty acid (e.g., linolenic acid, arachidonic acid, dihommo-γ-linolenic acid), whereby relatively less expression (“suppression”) of synthesis of PGE₂ results causing vasodilation and an increase of vascular flow in the treated area. Increased vascular flow improves mucus secretion and bicarbonate secretion, as protective factors against gastric and duodenal ulcers. These and other features, advantages and embodiments will be apparent to one of skill in the art from the following drawings and detailed description.

### BRIEF DESCRIPTION OF THE DRAWINGS

FIGS. 1A and B are Western blots showing COX-1 and COX-2 expression in transfected HAECs and HCASMCs. COX expression was measured 3 days after transfection of cells with Adnull or AdCOX-1 at 50, 100, 200, and 400 MOI. COX-1 expression in HAECs (A) and HCASMCs (B) was dependent on vector dose.

FIGS. 2A and B are Western blots similar to FIGS. 1A and B in which it is shown that COX-2 expression in HAECs (A) and HCASMCs (B) was not dependent on or induced by COX-1 overexpression.

FIGS. 3A and B are bar graphs showing the dose dependent generation of 6-keto PGF1α in HCASMC (FIG. 3A) and HAEC (FIG. 3B).

FIGS. 4A and B are bar graphs showing the dose dependent generation of PGE₁ in HCASMC (FIG. 4A) and HAEC (FIG. 4B).

FIGS. 5A and B are bar graphs showing the dose dependent generation of PGE₂ in HCASMC (FIG. 5A) and HAEC (FIG. 5B).

FIGS. 6A-F are bar graphs showing PGE₁ production in naïve (unfilled bars), Adnull-transfected (light shaded bars), and AdCOX-1-transfected (dark shaded bars) HAECs (FIGS. 6A-C) and HCASMCs (FIGS. 6D-F) after stimulation with no fatty acid (unstimulated) (FIGS. 6A and D), DGLA (FIGS. 6B and E), and AA (FIGS. 6C and F), respectively.

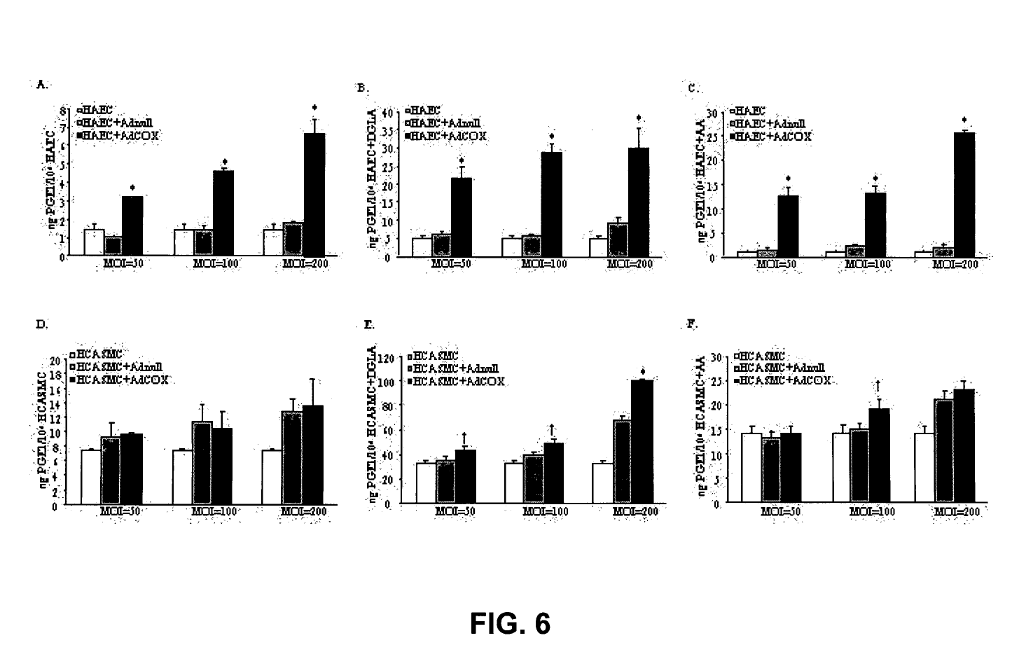

FIGS. 7A-F are bar graphs showing PGI₂ production in naïve (unfilled bars), Adnull-transfected (light shaded bars), and AdCOX-1-transfected (dark shaded bars) HAECs (FIGS. 7A-C) and HCASMCs (FIGS. 7D-F) after stimulation with no fatty acids (unstimulated) (FIGS. 7A and D), DGLA (FIGS. 7B and E), and AA (FIGS. 7C and F), respectively.

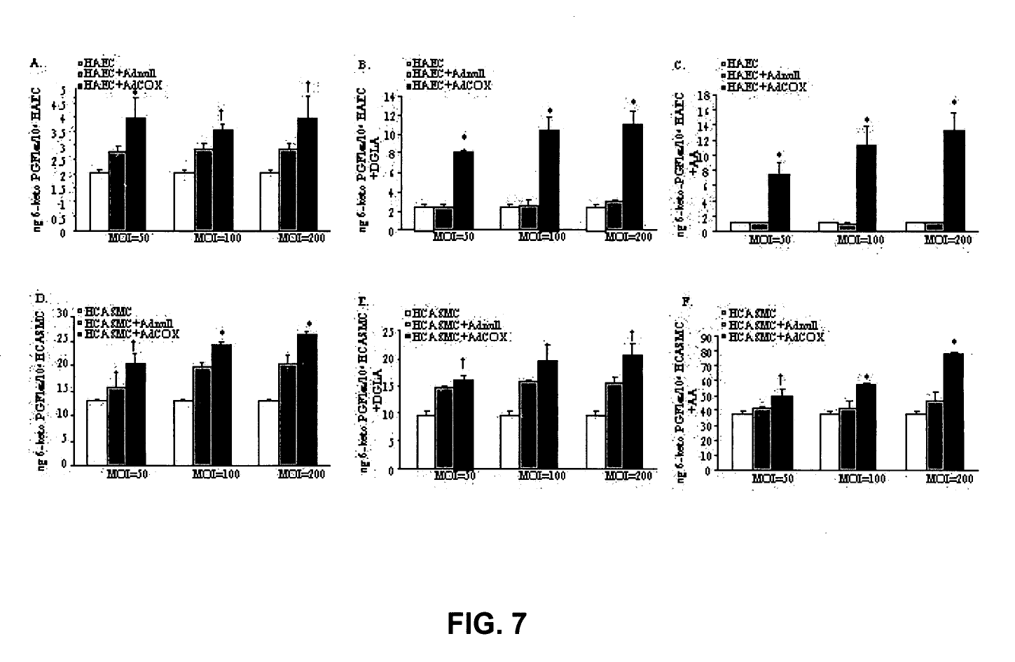

FIGS. 8A-F are bar graphs showing PGE₂ production in naïve (unfilled bars), Adnull-transfected (light shaded bars), and AdCOX-1-transfected (dark shaded bars) HAECs (FIGS. 8A-C) and HCASMCs (FIGS. 8D-F) after stimulation with no fatty acid (unstimulated) (FIGS. 8A and D), DGLA (FIGS. 8B and E), and AA (FIGS. 8C and F), respectively.

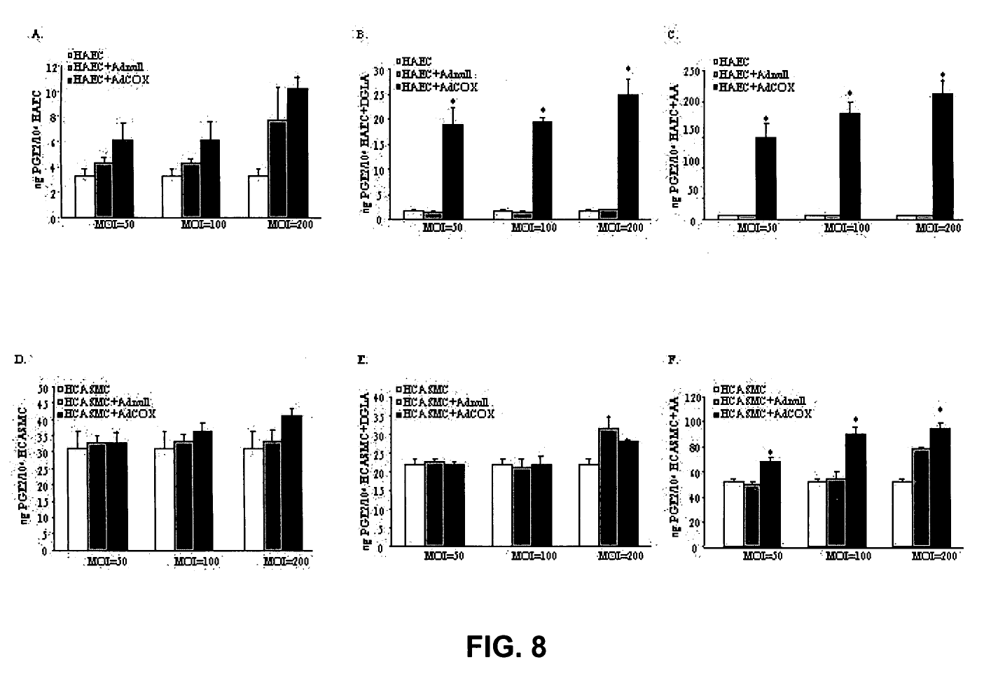

### DETAILED DESCRIPTION OF PREFERRED EMBODIMENTS

Studies were carried out to evaluate the effect of COX-1 dependent arachidonic acid (AA) metabolic pathway on PGE₁ biosynthesis in vitro and in vivo. It was discovered that COX-1 overexpression significantly increased PGE₁ level in both human aortic endothelial cell (HAEC) and human coronary artery smooth muscle cells (HCASMC). A positive effect of COX-1 on PGI₂ and PGE₂ production was also observed. In studies described in more detail in the Examples which follow, COX-1 was locally delivered to balloon-injured carotid arteries of New Zealand-White Rabbits. Ex vivo data were also obtained which illustrate that COX-1 preferentially stimulated PGI₂ production and meanwhile augmented PGE₁ and PGE₂ level.

It was further investigated whether cyclooxygenase-1 (COX-1) gene transfer can enhance expression of prostaglandins in addition to prostacyclin in vitro and in vivo, the prostaglandin expression profiles of human endothelial and smooth muscle cells in vitro and in balloon-injured carotid arteries of New Zealand White rabbits in vivo after COX-1 transfection and fatty acid stimulation were analyzed. COX-1 gene transfer followed by dihommo-γ-linolenic acid (DGLA) stimulation favorably enhanced PGE₁ and PGI₂ production and relatively suppressed PGE₂ production.

General Methods and Materials

Construction of an Adenoviral Vector Expressing Cyclooxygenase-1

Full-length human COX-1 cDNA (25 kb in size) was cloned into a replication deficient adenoviral vector. This vector contains a cytomegalovirus (CMV) early promoter, which drives the constitutive expression of COX-1. Adnull virus (30 kb), an identical adenoviral vector without any foreign gene, was also constructed, as previously described³⁰. AdCOX-1 and Adnull viruses were then purified. Viral particle concentrations and plaque forming units (PFU) were determined by both A260/280 ratio (Introgen) and plaque assays, using known techniques.

Cells culture and Adenoviral Transfection

Human aortic endothelial cells (HAEC, Cascade Biologics) were grown in conditional medium 200 supplemented with low serum growth supplement and PSA (Cascade Biologics). Human coronary artery smooth muscle cells (HCASMC, Cascade Biologics) were grown in the conditional medium 231 supplemented with smooth muscle differentiation supplement and PSA (Cascade Biologics). Both cell lines were maintained at 37° C. incubators containing 5% CO₂. Cells between passages 4-6 were utilized in the study.

Twenty-four hours prior to viral infection, HAEC and HCASMC were plated into 24-well tissue culture plates. Confluent monolayers were subsequently infected with AdCOX-1, Adnull and medium alone (mock) at multiplicity of infection (MOI) 50, 100, 200 and 400, respectively. After 6 hour postinfection, viruses were removed and cells were washed once with fresh mediums. Infected cells were then maintained in the growth condition at 37° C. In each infection, four or six individual wells of normal cells were infected with same viral concentration and at least three independent infections were conducted in each cell type.

Prostaglandin I₂ (PGI₂) measurement

Three days after viral infection, HAEC or HCASMC were supplied with fresh medium 200 or 231 containing 20 μM arachidonic acid and incubated at 37° C. for 45 min. Supernatants were collected and stored at −20° C. Cell numbers were counted by a cell counter (Coulter Electronics).

For the animal study, 3 days after local gene delivery, normal and viral infected carotid arteries were harvested from New Zealand-White rabbits. Artery ring (about 2 cm long) was cut open to expose the lumen and further cut into 6 small pieces. Artery pieces were washed three times with Dulbecco's modified eagle medium (DME, Invitrogen) and incubated with DME medium supplying with 50 μM arachidonic acid and 2% FBS at 37° C. for 45 min. Subsequent to supernatant collection, each artery piece was weighted and stored at liquid nitrogen.

PGI₂ production in supernatants was determined by using 6-keto prostaglandin F1a EIA kits (Cayman chemical). Previous to assay, individual samples were diluted to proper dilution with EIA buffer (provided by the manufacturer). EIA assays were carried out according to the manufacturer's instruction. EIA plates were read using a microplate reader at 415 nm (Bio-Rad). Each sample was assayed in duplicate.

Prostaglandin E1 (PGE₁) Prostaglandin E₂ (PGE₂) and Measurement

PGE₁ production was determined using prostaglandin E₁ enzyme immunoassay kits (Assay Design Inc). PGE₂ level in supernatants was quantified using monoclonal prostaglandin E₂ EIA kits (Cayman chemical). The assays were performed in accordance with manufacturer's protocols.

Preparation of AdCOX-1 and ADnull Infected Cytoplasmic Extracts

Three days after AdCOX-1 and Adnull infection, HAEC or HCASMC were washed twice with ice cold PBS. Cells were scraped with cell scrapers and centrifuged at 1500 rpm at 4° C. for 5 min to remove supernatant. Cell pellets were resuspended in isotonic buffer (25 mM Tris pH7.6; 1.5 mM MgCl₂; 10 mM KCl; 250 mM sucrose; 10 μg/ml leupeptin; 10 μg/ml aprotinin; 1 mM PMSF; 10 μM NaVO₄ and 1 μM NaF) and subsequently lysed by 0.5% NP-40. The extent of cell lysis was determined by visually inspecting the cells with a microscope. Cell lysates were centrifuged to remove cellular debris and protein concentrations were measured using a Bio-Rad protein determination kit.

Western Blot Analysis

Thirty microgram (30 μg) of total protein was mixed with an equal volume of Laemmli sample buffer (Bio-Rad) and fractionated in 10%-20% SDS-PAGE. Following electrophoresis, separated proteins were transferred into a PVDF membrane (Bio-Rad) at 30V overnight at 4° C. The membrane was blocked with TBS buffer containing 0.5% non-fat milk and probed with COX-1 (1:1000) or COX-2 (1:1000) mouse monoclonal antibodies (Santa Cruze Biotechnology). The antibody signals were visualized by ECL detection (Pierce) followed by exposure of the membrane to chemiluminescence film (Amersham). To verify equal loading of each protein sample, membranes were stripped after chemiluminescent detection and reprobed with β-actin (1:5000) monoclonal antibody (Sigma).

### EXAMPLE I

### COX-1 Promotes PGE₁ in Vascular Cells In Vitro and In Vivo

To evaluate the potential of the vascular smooth muscle cells (VSMC) and vascular endothelial cells (EC) to synthesize PGE₁ which possesses bioactivities similar to those of PGI₂ but longer half-life, we infected human aortic EC and coronary VSMC for 6 h with AdCOX-1, Adnull, and medium alone (mock). Seventy-two hours after infection, the cells were treated with 50 μM arachidonic acid at 37° C. for 45 min. Supernatants were then collected and productions of prostanoids were measured by EIAs (Tables 1 and 2).

TABLE 1
Prostanoids Released from Aortic EC at 72 Hour Postinfection

| Prostanoids | | Adnull | AdCOX-1 |
|---|---|---|---|
| (ng/10⁶cells) | Medium | (MOI = 200) | (MOI = 200) |
| PGI₂ | 0.54 ± 0.043 | 0.83 ± 0.078 | 12.45 ± 0.47 |
| PGE₁ | 2.24 ± 0.41 | 1.77 ± 0.31 | 16.13 ± 1.67 |
| PGE₂ | 2.45 ± 0.52 | 2.30 ± 0.58 | 55.12 ± 4.98 |

TABLE 2
Prostanoids Released from Coronary VSMC at 72 Hour Postinfection

| Prostanoids | | Adnull | AdCOX-1 |
|---|---|---|---|
| (ng/10⁶cells) | Medium | (MOI = 200) | (MOI = 200) |
| PGI₂ | 52.58 ± 3.00 | 74.25 ± 4.00 | 114.4 ± 3.81 |
| PGE₁ | 9.57 ± 0.80 | 16.66 ± 2.45 | 25.59 ± 1.14 |
| PGE₂ | 55.34 ± 4.1 | 79.96 ± 9.90 | 104.74 ± 0.93 |

Seventy-two hours after treatment with AdCOX-1, Adnull (AdCOX-1 minus gene), and medium alone, human aortic endothelial cell (HAEC) and human coronary artery smooth muscle cell (HCASMC) were stimulated with 50 μM arachidonic acid followed by EIA of PGE₁ as well as PGI₂ and PGE₂ in the supernatants. Compared to treatment with Adnull, PGE1, PGI₂ and PGE₂ increased considerably in AdCOX-1 treated HAEC. Enhanced productions of those prostaglandins were also achieved in AdCOX-1 treated HCASMC and baseline levels of PGE₁, PGI₂ and PGE₂ in HCASMC were higher than those of HAEC. The augments of PGE₁, PGI₂ and PGE₂ in both cell types were in AdCOX-1 dose-dependent manner. Furthermore, effective AdCOX-1 gene transfer did not cause COX-2 induction in HAEC and HCASMC.

Balloon Injury and COX-1 Transfection of Carotid Arteries in New Zealand White Rabbits

Balloon injury and local delivery of the COX-1 gene to carotid arteries of New Zealand White (NZW) rabbits were done according to a protocol approved by the animal committee at the University of Texas Houston Health Science Center. As described previously³¹, rabbit anesthesia was performed prior to each surgery. In brief, a no. 4 catheter introducer was introduced into the right femoral artery of anesthetized rabbits. Heparin at a dose of 150 units/kg was then administered to prevent arterial thrombosis during angioplasty. A balloon catheter (2.5×20 mm, Baxter Healthcare Corporation) was inserted into the femoral introducer and advanced to the right carotid artery with the assist of a guide wire under fluoroscopy. Balloon angioplasty was performed using 5 inflations to 8 atm for 30 second each time. One minute reperfusion was allowed between inflations. After angioplasty, the balloon was retracted 15 mm, and the proximal end of the damaged artery was ligated over the tip of the deflated angioplasty catheter with 2-0 silk over umbilical tape, to prevent further injury. All blood was removed from the damaged artery by repeated flushing with saline through the wire port of the angioplasty catheter. Then the distal portion of the damaged artery was ligated. After removing the remaining saline, 1×10¹⁰ PFU/ml AdCOX, Adnull or 1 ml of PBS were gradually introduced into the isolated artery, respectively. After incubating the virus in the artery for 30 min, the artery was flushed to remove virus or PBS and washed with saline. Finally, all incisions were repaired and the rabbits were monitored until they had recovered from anesthesia. Upon completing of surgery, the cut regions of rabbit were repaired and rabbits were recovered.

It was investigated whether injured carotid arteries in NZW rabbits were capable of producing PGE₁, employing the above-described method. Three days after balloon injury and local AdCOX-1 (1×10¹⁰ PFU) or Adnull (1×10¹⁰ PFU) delivery in New Zealand-White (NZ) rabbit's carotid arteries, levels of PGE₁, PGI₂ and PGE₂ were measured. AdCOX-1 transduced arteries showed significantly increased induction of prostacyclin (PGI₂) (n=5 produced 1,925±511 pg PGI₂ (measured as 6-keto PGF1α)/mg wet weight compared to Adnull treated arteries (n=5 produced 680±253 pg PGI₂/mg P<0.005, p≦0.02). PGE₂ level, albeit 5-10-fold lower than PGI₂, were also enhanced by COX-1 transduced arteries (n=4, 232±108 pg PGE₂/mg arteries) compared with control Adnull over-expressed arteries (99±58 pg PGE₂/mg arteries). COX-1 overexpression coincided with PGE₁ synthesis. Carotid arteries treated with AdCOX-1 demonstrated markedly higher secretion of PGE₁ than Adnull (n=4, P<0.05, 306±107 pg PGE₁/mg arteries compared to Adnull treated arteries (n=5, 148±58 pg PGE₁/mg arteries) and mock (PBS) treated arteries (n=4, 306±107 pg PGE₁/mg arteries vs. 139±30 pg/mg arteries) treated groups. Increased tissue levels of cAMP were also detected in COX-1 overexpressing arteries.

From these studies, also further below, it was concluded that, in addition to producing PGI₂, both aortic EC (HAEC) and coronary VSMC (HCASMC) are capable of synthesizing the vasoprotective eicosanoid PGE₁. PGE₁ was also synthesized in vivo in arteries and its synthesis was further enhanced by COX-1 gene transfer. Levels of PGE₂ are unexpectedly low, which may account in part for the limited inflammatory response reported previously after vascular COX-1 gene transfer in vivo.

COX-1 Proteins are Abundantly Expressed in HAEC and HCASMC

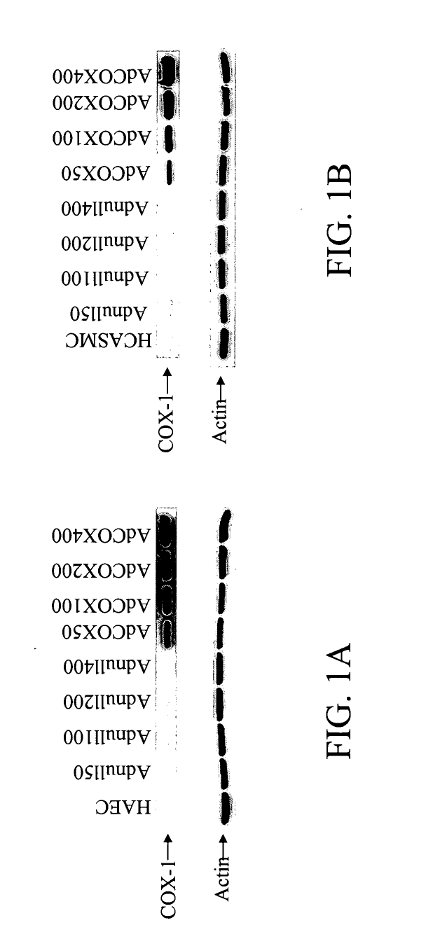

Western blots were initially utilized to estimate cycloxoygenase-1 uptake in both endothelial and smooth muscle cells. Relatively low doses (MOI=50 and 100) and high doses (MOI=200 and 400) of AdCOX-1 or Adnull (AdCOX-1 minus gene) were utilized to carry out the infection in HAEC and HCASMC. Preliminary time course studies (data not shown) suggested that both types of cells were effectively infected with viruses and COX-1 protein expression peak was reached during 72-96 hour postinfection. FIG. 1 shows that COX-1 overexpressions in both HAEC and HCASMC are dose dependent. By comparison, Adnull infected cells only exhibit a constant minimal level of COX-1 protein. In FIG. 1, dose dependent expression of cyclooxygenase-1 in HAEC (A) and HCASMC (B) are shown. Three days after Adnull or AdCOX-1 infection (MOI=50, 100, 200 and 400, respectively), HAEC and HCASMC were harvested and lysed. Thirty micrograms of cell lysates from each sample were subjected to electrophoresis followed by western blot analyses of COX-1 expression as described in methods. Reprobing the same membrane with β-actin antibody was performed to compare the difference of protein amount loaded in each sample. Results are representative of three different experiments.

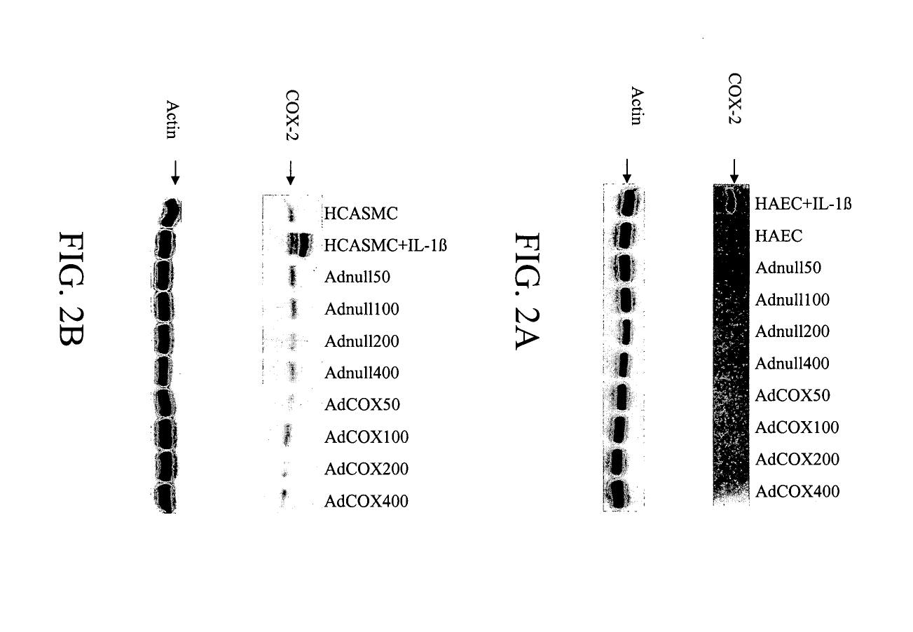

Due to impending side effects of COX-2 and its inducible property, it was investigated whether overexpressing COX-1 in HAEC and HCASMC might trigger COX-2 production. Same cell lysates used in COX-1 analyses were applied in COX-2 Western blot. Repeated experiment results verified that COX-2 protein was not detected in AdCOX-1 overexpressing HAEC (FIG. 2A). In HCASMC, basal level of COX-2 protein was observed in normal cells and in Adnull or AdCOX-1 overexpressing cells (FIG. 2B). To evaluate whether COX-2 induction in both HAEC and HCASMC could be achieved; normal cells were treated with interleukin-1β (IL-1β, 10 ng/ml) for 24 hour. COX-2 immunoblot experiments demonstrated that IL-1β was capable of prompting COX-2 induction in both cell types. In FIG. 2, the effect of COX-1 overexpression on COX-2 induction in HAEC (A) and HCASMC (B) is shown. Thirty micrograms of protein from the same batch of cell lysates prepared for COX-1 western blot were utilized for COX-2 detection. Western blot analyses of COX-2 expression were described in methods. To assess whether COX-2 could be stimulated in HAEC and HCASMC, interleukin-1β (10 ng/ml) treated cell lysates were also applied to COX-2 western blots. Reprobing the same membrane with β-actin antibody was performed to compare the difference amount protein amount loaded in each sample. Results are representative of two different experiments. Taken together, these data suggested that effective COX-1 gene transfer in HAEC and HCASMC did not sensitize COX-2 expression.

Prostaglandin I₂ Production was Increased in AdCOX-1 Infected HAEC and HCASMC

Having established recombinant COX-1 was successfully transferred and overexpressed in HAEC and HCASMC, we next employed experiments to assess whether COX-1 acts as an effective catalyst in COX pathway in both cells. Since COX-1 catalyzes the rate-limiting step in converting arachidonic acid to prostaglandins. Prostaglandins generated from arachidonic acid metabolism are best indicators of effects of COX-1 enzymatic activity.

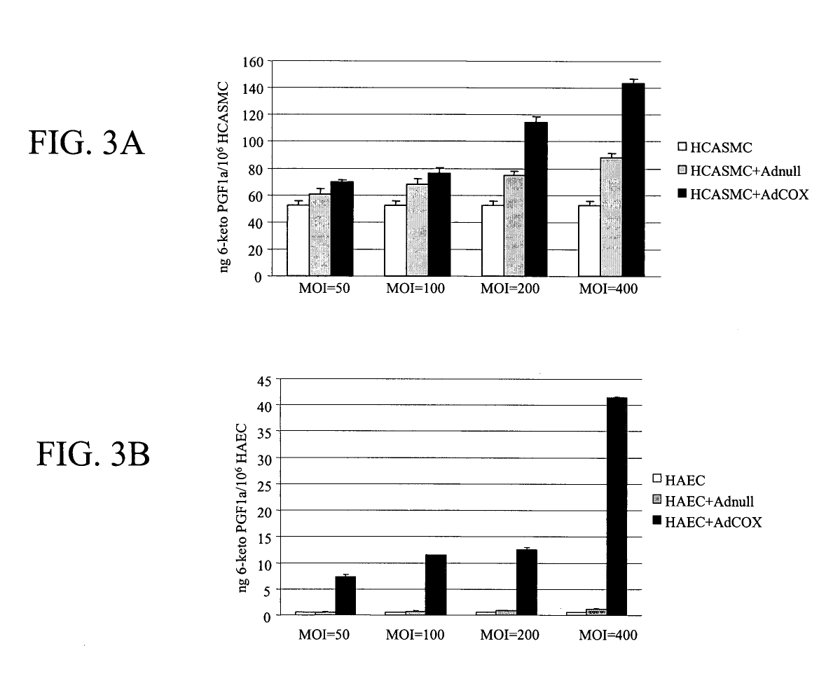

One of the prostaglandins-PGI₂ biosynthesis has been widely linked to COX-1 enzymatic activity in various studies. As a result, we performed immuno-absorbent assays to quantify PGI₂ stable metabolite; 6-keto PGF1α; in supernatants. After providing cells with saturating amount of exogenous arachidonic acid (50 μM), 6-keto PGF1α productions in medium (mock-infected), Adnull and AdCOX-1 infected cells were measured. In HCASMC, an increased level of 6-keto PGF1α was detected in AdCOX-1 infected cells compared with those infected with same dose of Adnull virus (FIG. 3A). Much more significant induction of 6-keto PGF1α in AdCOX-1 infected HAEC was achieved. The level of 6-keto PGF1α released from AdCOX-1 transduced HAEC was over 10-fold (range from 12 to 35 fold higher) than that of same dose of Adnull (FIG. 3B). It should be noted that 6-keto PGF1α production in both HAEC and HCASMC was in a dose-dependent manner and active normal HAEC makes less 6-keto PGF1α than HCASMC. In FIGS. 3A and B, dose dependent generation of 6-keto PGF1α in HAEC and HCASMC is shown in the form of bar graphs. Confluent HCASMC (FIG. 3A) and HAEC (FIG. 3B) in 24-well culture plates were infected with medium alone, Adnull or AdCOX-1 at MOI=50, 100, 200 and 400, respectively. Seventy-two hour postinfection, the cells were incubated with 50 μM of arachidonic acid for 45 minutes at 37° C. Subsequently, the supernatants were collected and the cell numbers in each well were counted. 6-keto PGF1α production was measured by EIA assays. Each MOI bar represents the mean±SD of four individual cell samples. Three independent experiments were performed. Significant difference of 6-keto PGF1α in AdCOX infected cells vs. Adnull infected cells was calculated by t-test (*P<0.005, #P<0.05).

COX-1 Promotes PGE₁ Productions in HAEC and HCASMC

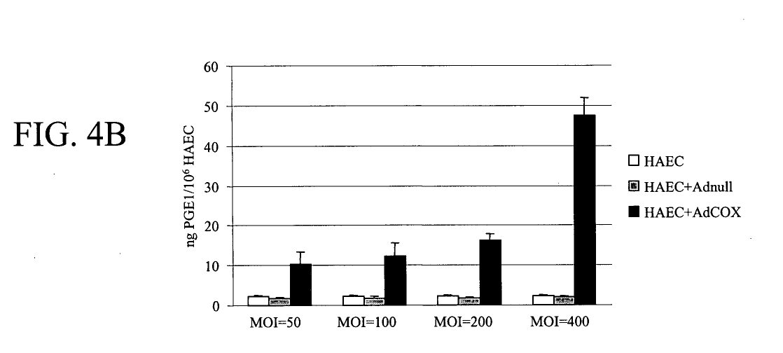

Recently, a number of investigations have identified PGE₁ as a potent agonist for deterring thrombus formation. PGE₁ is the major metabolite of dihommo-γ-linolenic acid via cyclooxygenase pathway. Little is known as to whether COX-1 mediated arachidonic acid metabolism could boost PGE₁ production in vascular endothelial and smooth muscle cells. To gain insight as to COX-1 signaling in this regard, we examined the PGE₁ induction in AdCOX-1 overexpressed HAEC and HCASMC. As shown in FIG. 4B, following treatment with arachidonic acid, AdCOX-1 infected HAEC (72 hour postinfection) resulted in considerable generation of PGE₁. By comparison with Adnull virus infected cells, the PGE₁ level was increased up to 24 fold at MOI 400. Additionally, PGE₁ produced in HAEC slightly exceeded PGI₂ when using the same dose of AdCOX-1. PGE₁ level in AdCOX-1 infected HCASMC was also enhanced (FIG. 4A) compared with those of Adnull infected HCASMC, but not as high as in HAEC. In FIGS. 4A and B, dose dependent generation of PGE1 in HAEC and HCASMC is shown. Confluent HCASMC (FIG. 4A) and HAEC (FIG. 4B) in 24-well culture plates were infected with Adnull and AdCOX-1 at MOI=50, 100, 200 and 400, respectively. Three days postinfection, the cells were incubated with 50 μM of arachidonic acid for 45 minutes at 37° C. Subsequently, the supernatants were collected and the cell numbers in each well were counted. PGE₁ production was measured by EIA assays. Each bar represents the mean±SD of four individual cell samples. Three independent experiments were performed. Significant difference of PGE₁ in AdCOX infected cells vs. AdRR infected cells was calculated by t-test (*P<0.005, #P<0.05).

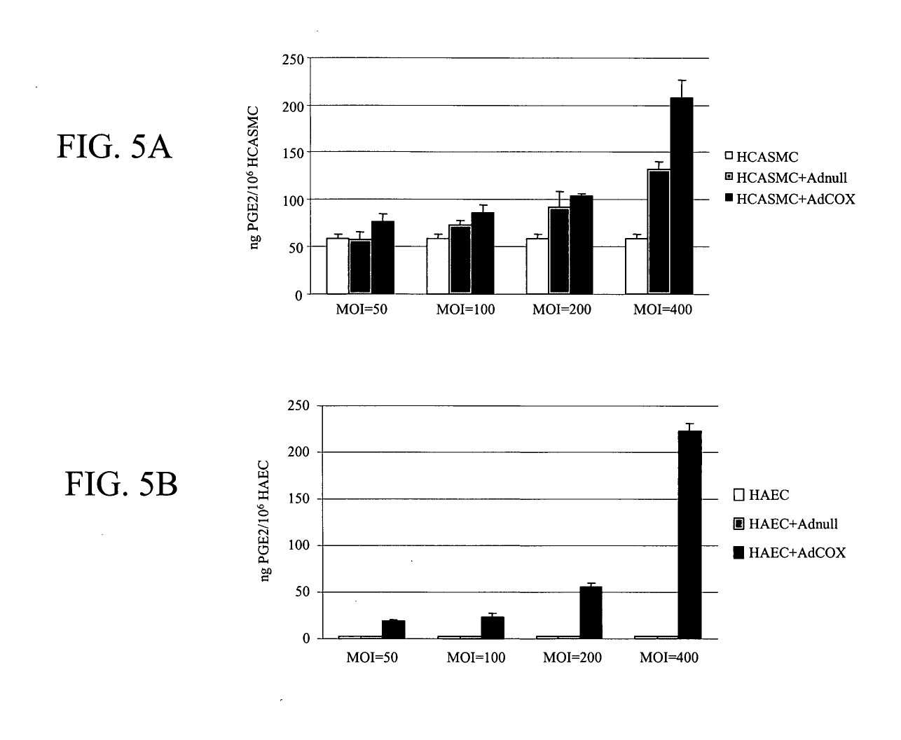

COX-1 gene transfer has been recognized to cause considerable induction of PGE₂. Therefore, we tested the PGE₂ level coincident with COX-1 overexpression following AA treatments. The same experimental systems were used to detect PGE₂ expression in HAEC and HCASMC. Again, dose dependent enhancement of PGE₂ were observed in AdCOX-1 infected cells (FIG. 5). AdCOX-1 infected HAEC (from MOI 50 to 400) produced 8.8˜91 fold higher PGE₂ than Adnull infected HAEC. Similar to PGE₁ and PGI₂ productions, much less increase of PGE₂ was detected in AdCOX-1 infected HCASMC vs. Adnull HCASMC. It appears that in addition to the common AA metabolites PGI₂ and PGE₂, COX-1 overexpression was associated with enhanced expression of PGE₁ in the vascular system. In FIGS. 5A and B, dose dependent generation of PGE₂ in HAEC and HCASMC is shown. Confluent HCASMC (FIG. 5A) and HAEC (FIG. 5B) in 24-well culture plates were infected with Adnull and AdCOX-1 at MOI=50, 100, 200 and 400, respectively. Three days postinfection, the cells were incubated with 50 μM of arachidonic acid for 45 minutes at 37° C. Subsequently, the supernatants were collected and the cell numbers in each well were counted. PGE₂ production was measured by EIA assays. Each bar represents the mean±SD of four individual cell samples. Three independent experiments were performed. Significant difference of PGE₂ in AdCOX infected cells vs. AdRR infected cells was calculated by student t-test (*P<0.005, #P<0.05).

Local Adcox-1 Gene Transfer to Carotid Artery Preferentially Increased PGI₂ Production

Having shown the effects of COX-1 on PGI₂, PGE₁ and PGE₂ productions in vascular endothelial and smooth muscle cells, we next conducted the animal study to test where in vivo results would be similar to those observed in vitro. 1×10¹⁰ PFU AdCOX-1 or 1×10¹⁰ PFU Adnull was transduced to balloon injured carotid arteries of New Zealand-White rabbits. Three days after gene delivery, carotid arteries were harvested and prostaglandins released from the lumen of arteries were measured.

As indicated in Table 3, baseline production of PGI₂ was higher than PGE₁ and PGE₂ after balloon injury of caroid arteries (PBS group, n=4). A 2.8 fold more PGI₂ was synthesized in AdCOX-1 transduced rabbits (n=5) comparing with those in Adnull treated ones (n=5). Meanwhile, a 2.3 fold rise of PGE₂ and a 2.2 fold increase of PGE₁ were as well coincident with enhanced expression of PGI₂ in AdCOX transduced arteries. Interestingly, carotid arteries exhibited predominant PGI₂ expression after local AdCOX-1 gene transfer. Incubation of artery pieces with arachidonic acid, under the same conditions used in the in vitro study, caused substantial secretion of PGI₂ (i.e., 8.2 times and 6.3 times increase) in AdCOX group compared with the same arteries that secreted PGE₂ and PGE₁, respectively.

TABLE 3
Prostaglandin Production in New Zealand Carotid Arteries

| | | Adnull | AdCOX |
|---|---|---|---|
| Prostaglandins | PBS | (1 × 10¹⁰ PFU) | (1 × 10¹⁰ PFU) |
| (pg PGs/mg artery) | (n = 4) | (n = 5) | (n = 5) |
| PGI₂ | 432 ± 135 | 680 ± 253 | 1925 ± 511*† |
| (6-keto PGF1a) | | | |
| PGE₂ | 94 ± 42 | 99 ± 58 | 232 ± 108*‡ |
| PGE₁ | 139 ± 30 | 148 ± 58 | 306 ± 107¶ |

Values are mean ± SD.
*P < 0.001, significantly different from PBS treated group
†P < 0.005 significantly different from AdRR-treated group
‡P < 0.05 significantly different from PBS and AdRR-treated groups
¶P < 0.05 significantly different from PBS and AdRR-treated groups

Discussion

In these studies, the effects of COX-1 overexpression on PGI₂, PGE₁ and PGE₂ synthesis were systematically examined. The resulting data demonstrated that both vascular endothelial and smooth muscle cells have the potential of synthesizing the vasoprotective prostaglandin PGE₁. In addition, PGE₁ synthesis was further enhanced by COX-1 gene transfer in vitro and in vivo. PGI₂ was a prevalent circulating prostaglandin resulting from adeno-vector induced COX-1 gene transfer in rabbit carotid arteries.

So far three series of prostaglandins have been defined^{32,33}. Prostaglandin series II and I are originated from linoleic acid. Prostaglandin series III are from γ-linolenic acid. The primary precursor of series II is arachidonic acid which comprises 5-15% of membrane fatty acid and which is the most common essential fatty acid in the cell membrane. The activation of phospholipase A2 releases arachidonic acid from phospholipid³⁴. Arachidonic acid then serves as the substrate for the cyclooxygenase catalyzed pathway that gives rise to prostacyclin, PGE₂ and other metabolites⁴. A similar cyclooxygenase pathway is present in series I prostaglandin synthesis^{32,35}. The primary precursor of series I prostaglandin (PGE₁) is dihommo-γ-linolenic acid (DGLA) generated from desaturation and elongation of dietary linolenic acid. DGLA serves as the substrate for subsequent cyclooxygenase enzymatic reactions resulting in the synthesis of PGE₁ and other derivatives. Furthermore, DGLA is unstable and rapidly desaturates to arachidonic acids³⁶⁻³⁸. Due to the availability and stability of DGLA on cell membranes, much interest has been focus on cyclooxygenase dependent AA metabolism that leads to the production of series II prostaglandins. In this study, we provide the evidence to show the positive impact of AA cascade on series I prostaglandins production and possible cross talk between prostaglandin series I and II. By supplying AA to COX-1 overexpressing cells, increased production of prostaglandin series II promotes PGE₁ production. As described in Example II, below, the prostaglandin profile of DGLA treated and COX-1 overexpressed cells was next examined.

Overlapping and diverse biological behaviors of cyclooxygenase isoforms have been reported by various studies^{3,18}. Cyclooxygenase isoforms exercise their functions mainly through regulating the synthesis of 20-carbon polyunsaturated fatty acid metabolites-prostaglandins. COX-1 dependent prostaglandins are widely recognized for their roles in platelet homeostasis and anti-atherosclerosis. Besides mediating brain and kidney function, COX-2 reliant prostaglandins are associated with some deleterious disease states such as Alzheimer's disease. Thus, COX-2 selective inhibitors are widely used in a variety of clinical applications to suppress unnecessary COX-2 activity^{19,39,40}. To eliminate the possibility that overexpressing COX-1 might initiate COX-2 induction in the above-described experimental condition, the COX-2 protein level was assessed using the same cytoplasmic extracts from cells that overexpress COX-1. The results of that investigation suggested that there was no COX-2 induction in response to AdCOX-1 overexpression in HAEC and HCASMC. However, minimal COX-2 expression was detected in active normal, adnull and AdCOX-1 infected HCASMC, which is possibly induced by growth factors supplied by culture medium.

Besides the crucial role of COX-1, generation of prostaglandins is associated with the activities of their terminal synthases such as PGI₂ synthase and PGE₂ synthase^{41,42}. The present results demonstrate that after arachidonic acid stimulation, the same number of active, normal HAEC (10⁶) produce 4 to 5 fold more PGE₂ than PGI₂ (FIGS. 3B and 5B). It might be that HAEC possesses more abundant PGE₂ synthase than PGI₂ synthase, however the quantity of enzyme is not likely to explain why the divergence between PGE₂ and PGI₂ production was augmented when using high doses of COX-1 (MOI=200 and 400). This might be attributed to the functional modulation of PGE₂ synthase by COX-1 that facilitates PGE₂ biosynthesis. Contrary to in vitro findings, PGI₂ instead of PGE₂ is the predominant product of COX-1 gene transfer in the balloon-injured rabbit artery model. It is known that the creation of endothelium damage by angioplasty disrupts blood vessel homeostasis and breaks the delicate state of relative vasodilation of the vessel wall. Gene transfer of COX-1 might accelerate the production of vasorelaxation factor PGI₂ that helps regain control of vessel tone. The evidence provided in this report suggests different mechanisms may be involved in regulating prostaglandin syntheses in vitro and in vivo. The relatively low level of arterial PGE₂ synthesis might also account for limited inflammatory response after COX-1 gene transfer. Further investigation is directed toward revealing key agonists that lead to upregulation of PGI₂ in vivo.

In terms of boosting or enhancing prostaglandin biosynthesis, COX-1 is much more potent in HAEC than in HCASMC, implicating other cellular factors that might add to COX-1 effects (such as increased gene transfer efficacy. As endothelial cells express a higher number of adenoviral receptors, viral carrier access is facilitated, as opposed to smooth muscle cells which have a very low number of adenoviral receptors). Interestingly, it appears that HCASMC has higher capacity of generating endogenous prostaglandins than HAEC upon arachidonic acid stimulation under growth condition. But COX-1 transfer only exerts minor effects on the expression of prostaglandins in HCASMC compared to HAEC. The Western blot data indicated that substantial amounts of COX-1 protein were expressed in HCASMC. This raises the possibility that endogenously formed PGI₂ might down-regulate PGI₂ biosynthesis or that some cellular factors might inhibit COX-1 enzymatic function in HCASMC. Alternatively, prostaglandin synthesis might be severely affected by the availability of their specific synthases.

Accumulating evidence points to PGE₁ as a valuable agent for promoting endothelial cells health and controlling smooth muscle cell proliferation⁴³. It has been reported that PGE₁ reduced neointimal hyperplasia after angioplasty and graft^{44,45}. Accordingly, PGE₁ is widely used to increase vascular blood flow in patients^{46,47}. However, there are few reports that show the effect of COX-1 induced AA metabolism on PGE₁ production in vascular systems. In light of the present observations, it is now suggested that COX-1 induced AA metabolism could have a direct action on PGE₁. In spite of lower expression level than PGE₂ in vitro, PGE₁ production exceeded PGI₂ in HAEC and also exceeded PGE₂ after local COX-1 gene delivery in vivo. As is known, prostaglandins operate as local hormones to exercise their spectrum of actions. The local production of PGE₁ by gene transfer of COX-1 might decrease or avoid at least some of the undesirable effects of PGE₂. Thus, this study has demonstrated an alternative pathway that increases availability of PGE₁ in vessel wall, which might support the vasodilating properties of PGI₂. COX-1 induced PGE₁ could also supplement or replace the diminished endogenous PGE₁ resulting from impaired endothelial function in atherosclerotic arteries. Without wishing to be limited to a particular theory, it is considered likely that PGE₁ exerts its bio-function through binding to prostaglandin E receptors and increasing cAMP. Ongoing studies by the inventors are directed at elucidation of this aspect.

### EXAMPLE II

### Selective Enhancement of PGE₁ and PGI₂ Production Relative to PGE₂ Prostaglandin Assays

Medium for both HAECs and HCASMCs was replaced with fresh medium alone 72 hours after adenoviral or mock treatment. Supernatants were collected 45 minutes later. Medium was replaced again with fresh medium containing either 20 μM DGLA or AA. Forty-five minutes later, supernatants were collected and stored at −20° C. until enzyme immunoassay (EIA) of PGE₁, PGI₂, and PGE₂ production. Cell numbers were counted in a Coulter counter. PGI₂ production in supernatants was quantified using a 6-keto PGF1α EIA kit (Cayman Chemical), PGE₂ production was quantified using a monoclonal PGE₂ EIA kit (Cayman Chemical), and PGE₁ was quantified using a PGE₁ immunoassay kit (Assay Design Inc), according to the manufacturers' instructions. EIA plates were read using a microplate reader at 415 nm (Bio-Rad). Each sample was assayed in duplicate. All other methods and materials were substantially as described in the General Methods and Materials above.

Enhancement of Beneficial PGE₁/PGE₂ Expression Profile by Fatty Acid Stimulation.

In brief, human aortic endothelial cells (HAECs) and human coronary artery smooth muscle cells (HCASMCs), cultured in vitro, were transfected with adenoviral COX-1 (AdCOX-1) or empty vector (Adnull) at varying multiplicities of infection (MOI) and stimulated with DGLA or arachidonic acid (AA). In vivo, balloon-injured carotid arteries of New Zealand White (NZW) rabbits were transfected with AdCOX-1 or Adnull 1×10¹⁰ PFU/ml). Three days later, the arteries were excised, cut into pieces, and incubated with DGLA or AA. Supernatants were processed for enzyme immunoassay of prostaglandins. After AdCOX-1 transfection, DGLA stimulation greatly enhanced PGE, production in endothelial cells, whereas AA stimulation lowered PGE₁ production and greatly enhanced PGE₂ production. The PGE₁-enhancing effects of AdCOX-1 transfection and DGLA stimulation were also noted in balloon-injured carotid arteries. The general methods and materials described above were employed in these studies, unless otherwise noted.

Balloon Injury and COX-1 Transfection of Carotid Arteries in New Zealand White Rabbits

Balloon injury and local delivery of the COX-1 gene to carotid arteries of NZW rabbits (n=24) were performed as described above. Eight rabbits were treated with AdCOX, and a like number were respectively treated with Adnull and PBS. At the time of balloon injury, all rabbits were similar in age: 18±3 months (AdCOX-1) vs. 17±4 months (Adnull) vs. 18±4 months (PBS).

Three days after local gene delivery, balloon-injured rabbits were sacrificed, and their carotid arteries were harvested as 2-cm-long artery rings. Each artery ring was cut open to expose the lumen and then into 6 small pieces. The pieces were washed 3 times with Dulbecco's modified Eagle's medium (DME; Invitrogen) and incubated with DMEM supplemented with 20 μM AA or DGLA and 2% FBS at 37° C. for 45 minutes. In each treatment group, 4 rabbits provided injured arteries for AA stimulation and 4 provided them for DGLA stimulation. Supernatants were collected and used for prostaglandin assays as described above. Each arterial piece was then dried and weighed.

Prostaglandin Production in Naïve Vascular Cells.

Naïve (nontransfected) HAECs and HCASMCs expressed 2- and 4-fold more PGE₁ than PGE₂, respectively (Table 4). Stimulation with DGLA increased PGE₁ production >3-fold in naive HAECs and >4-fold in HCASMCs. However, absolute PGE₁ production was 6-fold greater in HCASMCs than in stimulated naïve HAECs. Conversely, DGLA stimulation suppressed PGE₂ production in naïve HCASMCs and HAECs by one third to one half (35-50%). The ratios of PGE₁ to PGE₂ produced by DGLA-stimulated naïve HAECs and HCASMCs were 3 and 1.5, respectively. DGLA stimulation increased PGI₂ production only slightly in naive HAECs and lowered it by more than one fourth (28%) in naïve HCASMCs (Table 4).

Stimulation with AA suppressed PGE₁ production by approximately one fourth (24%) in naïve HAECs, but increased it >2-fold in naïve HCASMCs. Absolute PGE₁ production was almost 13-fold greater in stimulated naïve HCASMCs than in stimulated naïve HAECs. Conversely, AA stimulation increased PGE₂ production almost 2-fold in naïve HAECs and HCASMCs. AA stimulation increased PGI₂ production almost 3-fold in HCASMCs and by one third in HAECs (Table 4). PGE₁ production in AA-stimulated HAECs was >4 times lower and in AA-stimulated HCASMCs >2 times lower than in their DGLA-stimulated counterparts.

TABLE 4
Prostaglandin Production in HAECs and HCASMCs With/without Fatty Acids

| | HAECs | | | HCASMCs | | |
|---|---|---|---|---|---|---|
| | No | | | No | | |
| Prostaglandin | stimulation | AA | DGLA | stimulation | AA | DGLA |
| PGI₂ | 2.03 ± 0.12 | 2.70 ± 0.13 | 2.15 ± 0.33 | 13.02 ± 0.36 | 37.12 ± 2.54 | 9.30 ± 1.09 |
| PGE₁ | 1.45 ± 0.3 | 1.10 ± 0.41 | 5.00 ± 0.51 | 7.40 ± 0.29 | 14.04 ± 1.5 | 32.34 ± 2.41 |
| PGE₂ | 3.29 ± 0.50 | 6.17 ± 0.83 | 1.55 ± 0.37 | 31.00 ± 5.41 | 51.8 ± 2.46 | 22.05 ± 1.45 |

All data (ng/10⁶ cells) are expressed as mean ± SD for 4 experiments.
AA = arachidonic acid; DGLA = dihommo-γ-linolenic acid; HAECs = human aortic endothelial cells;
HCASMCs = human coronary artery smooth muscle cells.

PGE₁ Production in Transfected Vascular Cells.

Preliminary time-course studies established that COX-1 protein expression peaked at 72-96 hours after AdCOX-1 transfection in both HAECs and HCASMCs (data not shown). As shown by Western blot analysis, AdCOX-1-transfected HAECs and HCASMCs expressed abundant COX-1 protein in a dose-dependent manner (FIGS. 1A and B). By comparison, Adnull-transfected cells expressed minimal, though constant, amounts of COX-1. There was no cross-activation of COX-2 in the AdCOX-1-transfected cells (FIGS. 2A and B). Induction of COX-2 expression in both HAECs and HCASMCs appeared to be effectively controlled by stimulation with IL-1β (10 ng/ml for 24 hours) (FIGS. 2A and B).

Compared with unstimulated (naïve) HAECs, unstimulated HAECs transfected with AdCOX-1 produced 3- to 4-fold more PGE₁. PGE₁ production was further enhanced 3- to 4-fold more by AA stimulation and 4- and 7-fold more by DGLA stimulation. The highest PGE₁ production occurred in HAECs transfected with AdCOX-1 at MOI 100 and stimulated with DGLA.

Compared with unstimulated naïve HCASMCs, unstimulated HCASMCs transfected with AdCOX-1 produced 30-80% more PGE₁. AdCOX-1- and Adnull-transfected cells produced similar amounts of PGE₁. Arachidonic acid stimulation of AdCOX-1-transfected cells enhanced PGE₁ production by one half to four fifths (50-80%); DGLA stimulation enhanced it 4- to 7-fold. Absolute PGE₁ production was 2.5- to 4.5-fold higher in AdCOX-1-transfected HCASMCs stimulated with DGLA than in those stimulated with AA.

PGI₂ Production in Transfected Vascular Cells

Compared with unstimulated naïve HAECs, unstimulated HAECs transfected with AdCOX-1 produced 75-95% more PGI₂. PGI₂ production was further enhanced 2- to 3-fold more by either AA or DGLA stimulation. Absolute PGI₂ production by AdCOX-1-transfected HAECs was not differentially altered by stimulation with DGLA versus AA.

Compared with unstimulated naïve HCASMCs, unstimulated HCASMCs transfected with AdCOX-1 produced 1.5- to 2-fold more PGI₂. PGI₂ production was further enhanced 2.4- to 3-fold more by AA stimulation but suppressed by one fifth to one fourth (20-25%) by DGLA stimulation. Absolute PGI₂ production was 3-4 times greater in AdCOX-1-transfected HCASMCs stimulated with AA than in those stimulated with DGLA.

PGE₂ Production in Transfected Vascular Cells

Compared with unstimulated naïve HAECs, unstimulated HAECs transfected with AdCOX-1 produced 1.8- to 3-fold more PGE₂. PGE₂ production was further enhanced 20- to 40-fold more by AA stimulation and 2.5- to 3-fold more by DGLA stimulation. Absolute PGE₂ production was 7-9 times greater in AdCOX-1-transfected HAECs stimulated with AA than in those stimulated with DGLA.

Compared with unstimulated naïve HCASMCs, unstimulated HCASMCs transfected with AdCOX-1 produced one fourth (25%) more PGE₂. PGE₂ production was further enhanced 2-fold by AA stimulation but not enhanced at all by DGLA stimulation. Absolute PGE₂ production was 2-4 times greater in AdCOX-1-transfected HCASMCs stimulated with AA than in those stimulated with DGLA.

In brief, PGE₂ production in AdCOX-1-transfected HAECs, as opposed to Adnull-transfected (control) HAECs, increased 13- to 14-fold after DGLA stimulation and 20- to 30-fold after AA stimulation. The same did not hold true for smooth muscle cells, as PGE₂ production did not increase in AdCOX-1-transfected HCASMCs after DGLA stimulation but did increase by three fifths (60%) after AA stimulation.

Differential Prostaglandin Production in Carotid Arteries After Balloon Injury, Transfection, and Fatty Acid Stimulation

The prostaglandin expression profiles of Adnull-transfected and mock-treated carotid arteries were similar and were not differentially altered by fatty acid stimulation (Table 5). In comparison, the prostaglandin expression profile of AdCOX-1-transfected arteries was differentially affected by fatty acid stimulation. As compared with the production in both transfected and mock-treated controls, PGE₁ production in AdCOX-1-transfected arteries increased 2-fold after AA stimulation and 4-fold after DGLA stimulation; PGI₂ production increased 2.8-fold after AA stimulation and 2-fold after DGLA stimulation; and PGE₂ production increased 2-fold after AA stimulation but decreased 2-fold after DGLA stimulation.

TABLE 5
Prostaglandin Production in Balloon-injured Carotid Arteries of New Zealand White Rabbits

| | No vector | | | Adnull | | | AdCOX-1 | | |
|---|---|---|---|---|---|---|---|---|---|
| | (PBS) | | | (1 × 10¹⁰ PFU) | | | (1 × 10¹⁰ PFU) | | |
| Prosta- | No | | | No | | | No | | |
| glandin | stimulation | AA | DGLA | stimulation | AA | DGLA | stimulation | AA | DGLA |
| PGI₂ | 430 ± 123 | 432 ± 135 | 443 ± 201 | 490 ± 249 | 500 ± 280 | 425 ± 46 | 608 ± 136 | 1480 ± 160* | 1054 ± 232* |
| PGE₁ | 6.59 ± 5.66 | 31 ± 19 | 47 ± 3.90 | 9.49 ± 5.91 | 48 ± 16 | 41 ± 19 | 20 ± 5.56† | 93 ± 30† | 180 ± 64‡ |
| PGE₂ | 62 ± 13 | 74 ± 42 | 51 ± 20 | 66 ± 27 | 70 ± 30 | 98 ± 13 | 68 ± 25 | 140 ± 80 | 73 ± 22 |

All data (pg/mg arterial tissue) are expressed as mean ± SD for 4 experiments.
*P < 0.005, AdCOX-1 vs. Adnull group with same treatment.
†P < 0.05, AdCOX-1 vs. Adnull group with same treatment.
‡P < 0.01, AdCOX-1 vs. Adnull group with same treatment.
AA = arachidonic acid; DGLA = dihommo-γ-linolenic acid; PFU = plaque-forming units.

Discussion

In these studies, the expression of prostaglandins PGE₁, PGI₂, and PGE₂ in endothelial and smooth muscle cells in vitro and in balloon-injured arteries of NZW rabbits in vivo in the presence or absence of DGLA or AA has been successfully profiled. It is demonstrated that vascular cells in vitro are capable of endogenous PGE₁ synthesis and that the ratio of PGE₁ to PGE₂ production is much greater in HAECs than in HCASMCs (0.44 vs. 0.23). These findings suggest not only that endothelial cells are a major source of PGE, in vascular systems, but also that homeostasis of the vascular wall, under physiological conditions, depends heavily on the endothelium's health and its capacity to produce PGE₁, given PGE₁'s ability to control proliferation, inhibit cell migration, and inhibit inflammation. It was also found that DGLA stimulation favorably enhanced PGE₁ production in both HAECs and HCASMCs (as indicated by PGE₁/PGE₂ ratios of 3.22 and 1.46, respectively), whereas AA stimulation did not (as indicated by ratios of 0.16 and 0.27, respectively). However, both fatty acids were capable of enhancing PGI2 production in HAECs (by 5% and 25%, respectively). Nevertheless, DGLA remains a much better alternative, given its stimulatory effect on PGE, and inability (unlike AA) to generate thromboxane A2 (TXA₂). It is of interest to note that these findings establish a simple biologic basis for the clinically and widely accepted notion that consuming fish and borage oil, both important natural sources of DGLA, can exert favorable vasoprotective and cardioprotective effects.

Also notable was the effect of COX-1 gene transfer alone on HAECs in vitro. COX-1 gene transfer alone at 50, 100, and 200 MOI resulted in substantially higher PGE₁ production than did DGLA stimulation alone. By providing in vitro evidence that the amount of DGLA stored in the membrane phospholipid layer of endothelial cells ensures the availability of more than enough enzyme substrate for the naturally expressed COX-1, this finding underscores the importance of COX-1 availability to the production of PGE₁. On the other hand, the increase in PGE₁ production seen after DGLA stimulation of naïve, nontransfected HAECs suggests that COX-1's kinetic preference can be shifted in favor of DGLA whenever more of this substrate becomes available.

The difference between PGE₁/PGE₂ ratios in naïve HAECs (0.44) and COX-1-transfected HAECs (0.52-0.75 at varying MOI) suggests that PGE₁ and PGE₂ production depends not only on the availability of a specific precursor and COX-1, but also on the availability and actions of different specific PG synthases. This in turn suggests the possibility that intricate, autocrine, regulatory mechanisms of PGE₁-PGE₂ interaction (e.g., activity of specific PGE₁ and PGE₂ synthases and autocrine interregulation of PGE₁ and PGE₂ production) ultimately favor PGE₁ over PGE₂ synthesis when COX-1 and DGLA become abundant. The potential importance of these mechanisms is highlighted by the finding that PGE₁/PGE₂ ratios in DGLA-stimulated, COX-1-transfected HAECs (1.15-1.46 at varying MOI) were significantly lower than in DGLA-stimulated HAECs (3.22). This proved that PGE₂ production was optimally suppressed in naïve nontransfected HAECs in which an adequate, accommodative ratio of COX-1/PGE₁ synthase was present. Thus, it is proposed that COX-1 gene transfer combined with DGLA stimulation as a means of favorably enhancing prostaglandin expression profiles can be further refined by combining COX-1 and PGE₁ synthase gene transfer, in order to achieve a more accommodative COX-1/PGE₁ synthase ratio closer to the one found in naïve HAECs.

Finally, one of the most interesting findings from these studies was the translation of the in vitro findings into a rabbit model in vivo. It was found that the only experimental treatment that strongly enhanced PGE₁ production in relation to PGE₂ production was COX-1 transfer and DGLA stimulation. The resulting PGE₁/PGE₂ ratio of 2.46 was significantly higher than the ratios in all other treatment groups, in which PGE₁/PGE₂ ratio invariably remained lower than 1. Combined local COX-1 gene transfer and oral DGLA administration may be of potential therapeutic use as an alternative treatment for prevention of restenosis after angioplasty, and may provide a means to improve blood flow in patients with peripheral and coronary heart disease.

The exemplary methods and results with COX-1, AA and DGLA are considered representative of how other COX isoforms, such as COX-2, could be likewise transferred to cells in vitro and in vivo, and how other fatty acid precursors could be used similarly to AA and DGLA for stimulation of PGE₁ production. It is also proposed that EPA, a precursor for series 3 prostaglandins, administered added to DGLA will enhance PGE₁ production will help preserve DGLA and block its metabolization to AA. The exemplary methods using vascular endothelial cells and smooth muscle cells are considered representative of methods employing gastric mucosal cells and submucosal cells, and that the present results for VECs and VSMCs are considered predictive of similar results that will be obtained with gastric mucosal and submucosal cells.

Without further elaboration, it is believed that one skilled in the art can, using the description herein, utilize the present invention to its fullest extent. The foregoing embodiments are to be construed as illustrative, and not as constraining the remainder of the disclosure in any way whatsoever. While the preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. For example, from the inventors' discovery that synthesis of PGE₁ in vascular (endothelial, smooth muscle, and other cells residing in vascular conduits) can be enhanced by gene transfer of COX-1, it can be readily appreciated that, by extension, similar compositions and methods can be employed for treating these and other cells by gene transfer of COX-1, COX-2, and other isoforms of this enzyme to achieve similar enhancement of PGE₁ production. Adenovirus is used as a representative example for establishing the feasibility of this approach, but any gene transfer vector composition and any method in the art, other ex vivo transduction methods of cells and conduits (vein grafts, stent grafts), and ex vivo/in vivo gene transfer of COX isoforms with or without administration of DGLA, other fatty acids, and other prostaglandin synthesizing enzymes (such as PGES and PGIS) and related enzymes to organs including the native kidneys, to allografts (e.g., heart, kidney) prior to their implantation in organ recipients, or to the central nervous system, including its supporting structures, is encompassed by the present disclosure and within the scope of the present invention. The disclosures of all patents, patent applications and publications cited herein are hereby incorporated herein by reference, to the extent that they provide materials and/or methods supplementary to those described herein.

### REFERENCES

1. Smith W L, Song I. The enzymology of prostaglandin endoperoxide H synthases-1 and -2. Prostaglandins Other Lipid Mediat. 2002;68-69: 115-28.
2. Tanabe T, Tohnai N. Cyclooxygenase isozymes and their gene structures and expression. Prostaglandins Other Lipid Mediat. 2002;68-69:95-114.
3. Crofford L J. Prostaglandin biology. Gastroenterol Clin North Am. 2001;30:863-76.
4. Garavito R M, DeWitt D L. The cyclooxygenase isoforms: structural insights into the conversion of arachidonic acid to prostaglandins. Biochim Biophys Acta. 1999; 1441:278-87.
5. Morita I. Distinct functions of COX-1 and COX-2. Prostaglandins Other Lipid Mediat. 2002;68-69: 165-75.
6. van der Donk W A, Tsai A L, Kulmacz R J. The cyclooxygenase reaction mechanism. Biochemistry. 2002;41:15451-8.
7. Garavito R M, Malkowski M G, DeWitt D L. The structures of prostaglandin endoperoxide H synthases-1 and -2. Prostaglandins Other Lipid Mediat. 2002;68-69:129-52.
8. Hinz B, Brune K. Cyclooxygenase-2—10 years later. J Pharmacol Exp Ther. 2002;300:367-75.
9. Weinberg J B. Nitric oxide synthase 2 and cyclooxygenase 2 interactions in inflammation. Immunol Res. 2000;22:319-41.
10. Simon L S. Role and regulation of cyclooxygenase-2 during inflammation. Am J Med. 1999;106:37S-42S.
11. Vane J R, Bakhle Y S, Botting R M. Cyclooxygenases 1 and 2. Annu Rev Pharmacol Toxicol. 1998;38:97-120.
12. Smith W L, DeWitt D L, Garavito R M. Cyclooxygenases: structural, cellular, and molecular biology. Annu Rev Biochem. 2000;69: 145-82.
13. Fort J. Celecoxib, a COX-2—specific inhibitor: the clinical data. Am J Orthop. 1999;28:13-8.
14. Tanaka A, Hase S, Miyazawa T, Ohno R, Takeuchi K. Role of cyclooxygenase (COX)-1 and COX-2 inhibition in nonsteroidal anti-inflammatory drug-induced intestinal damage in rats:
   relation to various pathogenic events. J Pharmacol Exp Ther. 2002;303: 1248-54.
15. Schlondorff D, Ardaillou R. Prostaglandins and other arachidonic acid metabolites in the kidney. Kidney Int. 1986;29:108-19.
16. Raskin J B. Gastrointestinal effects of nonsteroidal anti-inflammatory therapy. Am J Med. 1999;106:3s-12S.
17. Wu K K. Prostacyclin and nitric oxide-related gene transfer in preventing arterial thrombosis and restenosis. Agents Actions Suppl. 1997;48: 107-23.
18. Lettino M, Cantu F, Mariani M. Cyclo-oxygenase-1 and cyclo-oxygenase-2 and cardiovascular system. Dig Liver Dis. 2001;33:S12-20.
19. Turini M E, DuBois R N. Cyclooxygenase-2: a therapeutic target. Annu Rev Med. 2002;53:35-57.
20. Pruthi R S, Derksen E, Gaston K. Cyclooxygenase-2 as a potential target in the prevention and treatment of genitourinary tumors: a review. J Urol. 2003; 169:2352-9.
21. Singh B, Lucci A. Role of cyclooxygenase-2 in breast cancer. J Surg Res. 2002; 108:173-9.
22. Williams C S, Mann M, DuBois R N. The role of cyclooxygenases in inflammation, cancer, and development. Oncogene. 1999; 18:7908-16.
23. Wolfe L S. Eicosanoids: prostaglandins, thromboxanes, leukotrienes, and other derivatives of carbon-20 unsaturated fatty acids. J Neurochem. 1982;38: 1-14.
24. Dajani E Z, Wilson D E, Agrawal N M. Prostaglandins: an overview of the worldwide clinical experience. J Assoc Acad Minor Phys. 1991;2:23, 27-35.
25. Clapp L H, Finney P, Turcato S, Tran S, Rubin L J, Tinker A. Differential effects of stable prostacyclin analogs on smooth muscle proliferation and cyclic AMP generation in human pulmonary artery. Am J Respir Cell Mol. Biol. 2002;26: 194-201.
26. Newby A C, Southgate K M, Assender J W. Inhibition of vascular smooth muscle cell proliferation by endothelium-dependent vasodilators. Herz. 1992;17:291-9.
27. Weber A A, Zucker T P, Hasse A, Bonisch D, Wittpoth M, Schror K. Antimitogenic effects of vasodilatory prostaglandins in coronary artery smooth muscle cells. Basic Res Cardiol. 1998;93:54-7.
28. Paoletti P, Chiabrando C, Gaetani P, Castelli MG, Butti G, Martelli L, Rolli M. Prostaglandins in human brain tumors. J Neurosurg Sci. 1989;33:65-9.
29. Uotila P. The role of cyclic AMP and oxygen intermediates in the inhibition of cellular immunity in cancer. Cancer Immunol Immunother. 1996;43: 1-9.
30. Zoldhelyi P, McNatt J, Xu X M, Loose-Mitchell D, Meidell R S, Clubb F J, Jr., Buja L M, Willerson J T, Wu K K. Prevention of arterial thrombosis by adenovirus-mediated transfer of cyclooxygenase gene. Circulation. 1996;93:10-7.
31. Zoldhelyi P, Chen Z Q, Shelat HS, McNatt J M, Willerson J T. Local gene transfer of tissue factor pathway inhibitor regulates intimal hyperplasia in atherosclerotic arteries. Proc Natl Acad Sci USA. 2001;98:4078-83.
32. Horrobin D F. A new concept of lifestyle-related cardiovascular disease: the importance of interactions between cholesterol, essential fatty acids, prostaglandin E1 and thromboxane A2. Med Hypotheses. 1980;6:785-800.
33. Adam O. Cardiovascular effects of n-6 fatty acids. Klin Wochenschr. 1990;68:16-22.
34. Murakami M, Kudo I. Phospholipase A2. J Biochem (Tokyo). 2002; 131:285-92.
35. Kirtland S J. Prostaglandin E1: a review. Prostaglandins Leukot Essent Fatty Acids. 1988;32: 165-74.
36. Ziboh V A. The significance of polyunsaturated fatty acids in cutaneous biology. Lipids. 1996;31:S249-53.
37. Bezard J, Blond J P, Bernard A, Clouet P. The metabolism and availability of essential fatty acids in animal and human tissues. Reprod Nutr Dev. 1994;34:539-68.
38. Horrobin D F. Prostaglandin E1: physiological significance and clinical use. Wien Klin Wochenschr. 1988;100:471-7.
39. Krzystyniak K L. Current strategies for anticancer chemoprevention and chemoprotection. Acta Pol Pharm. 2002;59:473-8.
40. Subbaramaiah K, Dannenberg A J. Cyclooxygenase 2: a molecular target for cancer prevention and treatment. Trends Pharmacol Sci. 2003;24:96-102.
41. Tanabe T, Ullrich V. Prostacyclin and thromboxane synthases. J Lipid Mediat Cell Signal. 1995;12:243-55.
42. Urade Y, Watanabe K, Hayaishi O. Prostaglandin D, E, and F synthases. J Lipid Mediat Cell Signal. 1995;12:257-73.
43. Schror K, Weber A A. Roles of vasodilatory prostaglandins in mitogenesis of vascular smooth muscle cells. Agents Actions Suppl. 1997;48:63-91.
44. Komori K, Furuyama T, Shoji T, Kume M, Mori E, Yamaoka T, Sugimachi K. Inhibitory effect of prostaglandin E 1 on intimal thickening caused by poor runoff conditions in the canine autologous vein grafts. J Cardiovasc Pharmacol. 2001;38:686-92.
45. Motoyama Y, Seki J, Katoh Y, Nishio M, Yoshida K. Effect of TFC-612, a 7-thia prostaglandin E1 derivative, on intimal thickening after endothelial injury with balloon catheter in rats. Atherosclerosis. 1994; 108:159-65.
46. Fellows K E, Jr. Therapeutic catheter procedures in congenital heart disease: current status and future prospects. Cardiovasc Intervent Radiol. 1984;7: 170-7.
47. Linhart J. Prostaglandin therapy of peripheral occlusive arterial disease. Sb Lek. 1998;99:355-62.
48. Wu K K. Prostacyclin and nitric oxide-related gene transfer in preventing arterial thrombosis and restenosis. Agents Actions Suppl. 1997;48: 107-123.
49. Willis A L, Stone J K, Hart M, et al. Dietary fatty acids, prostaglandins, endoperoxidases and the prevention of thrombosis. In: Prostaglandins in Hematology. New York, N.Y.: Spectrum; 1977:371-410.
50. Kloeze J. Relationship between chemical structure and platelet-aggregation activity of prostaglandins. Biochim Biophys Acta. 1969; 187:285-292.
51. Cameron N E, Cotter M A. Comparison of the effects of ascorbyl gamma-linolenic acid and gamma-linolenic acid in the correction of neurovascular deficits in diabetic rats. Diabetologia. 1996;39: 1047-1054.
52. Zurier R B, Quagliata F. Effect of prostaglandin E 1 on adjuvant arthritis. Nature. 1971;234:304-305.
53. Zurier R B, Sayadoff D M, Torrey A B, et al. Prostaglandin E treatment of NZB/NZW mice. Arthritis Rheum. 1977;20:723-728.
54. Virgolini I, Li S R, Lupattelli G, et al. Effect of prostaglandin E1 on low density lipoprotein apo-B-receptor binding in human, rat and swine liver in vitro. Prostaglandins. 1991;42:81-93.
55. Salvatori R, Guidon P T, Jr., Rapuano B E, et al. Prostaglandin E1 inhibits collagenase gene expression in rabbit synoviocytes and human fibroblasts. Endocrinology. 1992; 131:21-28.
56. Marchesi S, Pasqualini L, Lombardini R, et al. Prostaglandin E1 improves endothelial function in critical limb ischemia. J Cardiovasc Pharmacol. 2003;41:249-253.
57. Raz A, Minkes M S, Needleman P. Endoperoxides and thromboxanes: structural determinants for platelet aggregation and vasoconstriction. Biochim Biophys Acta. 1977;488:305-311.
58. Laneuville O, Breuer D K, Xu N, et al. Fatty acid substrate specificities of human prostaglandin-endoperoxide H synthase-1 and -2: formation of 12-hydroxy-(9Z, 13E/Z, 15Z)-octadecatrienoic acids from alpha-linolenic acid. J Biol Chem. 1995;270: 19330-19336.
59. Levin G, Duffin K L, Obukowicz M G, et al. Differential metabolism of dihommo-gamma-linolenic acid and arachidonic acid by cyclo-oxygenase-1 and cyclo-oxygenase-2: implications for cellular synthesis of prostaglandin E1 and prostaglandin E2. Biochem J. 2002;365:489-496.
60. Johnson M M, Swan D D, Surette M E, et al. Dietary supplementation with gamma-linolenic acid alters fatty acid content and eicosanoid production in healthy humans. J Nutr. 1997;127:1435-1444.
61. Zurier R B, Rossetti R G, Jacobson E W, et al. gamma-Linolenic acid treatment of rheumatoid arthritis: a randomized, placebo-controlled trial. Arthritis Rheum. 1996;39: 1808-1817.
62. Fan Y Y, Chapkin R S, Ramos K S. Dietary lipid source alters murine macrophage/vascular smooth muscle cell interactions in vitro. J Nutr. 1996;126:2083-2088.
63. Kernoff P B, Willis A L, Stone K J, et al. Antithrombotic potential of dihommo-gamma-linolenic acid in man. Br Med J. 1977;2:1441-1444.
64. Fan Y Y, Ramos K S, Chapkin R S. Dietary gamma-linolenic acid suppresses aortic smooth muscle cell proliferation and modifies atherosclerotic lesions in apolipoprotein E knockout mice. J Nutr. 2001;131:1675-1681.
65. Doyle M J, Nemeth P R, Skoglund M L, Mandel K G. In vivo assessment of precursor induced prostaglandin release within the rat gastric lumen. Prostaglandins. 1989;38:581-97.
66. Fukuda T, Arakawa T, Torii Y, Nebiki H, Nakamura H, Kobayashi K. Roles of endogenous leukotrienes and prostaglandins in the healing of gastric ulcers induced by acetic acid in rats. Scand J Gastroenterol Suppl. 1989; 162:2-5.
67. Arvidsson S, Falt K, Haglund U. Gastric mucosal damage in sepsis—effects of pretreatment with a synthetic prostaglandin E1 analogue. Gut. 1985 26:1025-31.
68. Kawarada Y, Lambek J, Matsumoto T. Pathophysiology of stress ulcer and its prevention. II. Prostaglandin E1 and microcirculatory responses in stress ulcer. Am J. Surg. 1975 129:217-22.
69. Dajani E Z. Overview of the mucosal protective effects of misoprostol in man. Prostaglandins. 1987;33 Suppl: 117-29. Review.
70. Sankey S L, Friedman L S. Formulary management of antiulcer drugs: clinical considerations. Pharmacoeconomics. 1994 5:213-26. Review.

1. A method of enhancing production of PGE₁ in cells comprising:
introducing a recombinant cDNA encoding at least one cyclooxygenase isoform into said cells, such that cells overexpress said cyclooxygenase isoform; and
treating said overexpressing cells with at least one fatty acid substrate for said at least one cyclooxygenase isoform, whereby production of PGE₁ by said cells is enhanced.

2. The method of claim 1 wherein said cells comprise vascular endothelial cells.

3. The method of claim 1 wherein said cells comprise vascular smooth muscle cells.

4. The method of claim 1 wherein said cells comprise gastric mucosal cells or gastric submucosal cells.

5. The method of claim 1 wherein said at least one cyclooxygenase comprises COX-1.

6. The method of claim 1 wherein said at least one cyclooxygenase comprises COX-2.

7. The method of claim 1 wherein said at least one fatty acid substrate is chosen from the group consisting of linolenic acid, arachidonic acid, and dihommo-γ-linolenic acid.

8. The method of claim 1 wherein said step of treating said cells comprises in vivo administration to an individual in need thereof an amount of said at least one fatty acid substrate, effective to further enhance the synthesis of PGE₁ in said vascular cells.

9. The method of claim 8 wherein said step of treating said overexpressing cells comprises administering an amount of said at least one fatty acid substrate effective to produce a prostaglandin expression profile in said cells in which PGE₁ and PGI₂ production is increased relative to PGE2 expression.

10. The method of claim 9 wherein said administering comprises establishing a concentration of at least 20 μM dihommo-γ-linolenic acid in said cells.

11. The method of claim 1 wherein said step of introducing said recombinant cDNA into said cells comprises in vivo contacting of said cells, whereby in vivo production of PGE₁ in said contacted cells at said site is enhanced.

12. A method of treating a pathophysiological condition in an individual suffering therefrom comprising carrying out the method of claim 11 such that said condition is improved by said enhanced production of PGE₁.

13. The method of claim 12 wherein said condition comprises a cardiovascular condition chosen from the group consisting of vascular stenosis, thrombosis and inflammatory disease, and said cells comprise vascular cells.

14. The method of claim 12 wherein said condition comprises impaired renal function and said cells comprise vascular cells, wherein renal function in said individual is improved by said enhanced production of PGE₁ in said vascular cells.

15. The method of claim 12 wherein said condition comprises stroke and said cells comprise vascular cells, wherein said stroke is prevented in said individual, or the effects of stroke in said individual are lessened by said enhanced production of PGE₁ in said vascular cells.

16. The method of claim 12 wherein said condition comprises bronchoconstrictive disease and said cells comprise vascular cells, wherein said enhanced production of PGE₁ in said vascular cells stimulates adenylyl cyclase and local increase in cyclic AMP, whereby bronchodilation is induced in said individual.

17. The method of claim 12 wherein said condition comprises a renal or cardiac allograft in need of protection from vascular stenosis and said cells comprise vascular cells, and wherein said enhanced production of PGE₁ in said vascular cells provides a vasoprotective effect in said allograft.

18. The method of claim 12 wherein said condition comprises an angioplasty site at risk of restenosis and said cells comprise vascular cells, and wherein said enhanced production of PGE₁ in said vascular cells provides at least some protection from restenosis at said site.

19. The method of claim 11 wherein said condition comprises peripheral vascular disease and said cells comprise vascular cells, and wherein said enhanced production of PGE₁ in said vascular cells provides at least some improvement of blood flow in a vessel containing said vascular cells.

20. The method of claim 11 wherein said condition comprises coronary heart disease and said cells comprise vascular cells, and wherein said enhanced production of PGE₁ in said vascular cells provides at least some improvement of blood flow in the heart of the individual.

21. The method of claim 11 wherein said condition comprises peptic ulcer disease and said cells comprise gastric mucosal and/or submucosal cells, and wherein said enhanced production of PGE₁ in said mucosal and submucosal cells provides at least some vasodilation leading to increased blood flow.

22. The method of claim 21 wherein said increased blood flow is effective to improve mucus secretion and/or bicarbonate secretion in the gastrointestinal system of the treated individual.

23. A kit comprising:
a COX isoform transducing vector; and
at least one fatty acid substrate for said COX isoform in a pharmaceutically acceptable carrier.
